SEQUENCE LISTING (30 sequences)

SEQ ID NO: 1 - DNA, 8541 bp, Rice
gttgaagcatagatatctgataggccagtacttgctccactcgtgtggttaaacaaagaggggcacattattaggagtaatagacaaaaatcgccatagtttctagcctacgattcaaaaagagaaccacccaattcacttttgggcattgttcatcgacttgacttggactgccctgttatcagaattaggttcgctgggatgacatgatttaaaagcatcaatgatttgcacctaaaatgtgaatgacatattgacattcctactactgtagagagaaacatcattctgacaaaagaaacactacagaaaaacgaaatttcctccagcagttgcagagaggaacttttcatctctcccgtggtcttggatcctctatttcccacgttggctgaaatgcaagcttattctgaactgtccgtctggatgatgggcgagattcaggtaaaaaagaagcatggtgttcaggttgcatgcgattcaatcatctatgtcatgttcatgtggaaaaaatttattgagctggcccaagtgcctgctccagtgttcctggactcaattataaccaaatcatactagctagagtagttgacttcagcgatgccggctgcgctacactcatcgaccagcagtgttgagaacccatcatagctagctcaatgtgactttaggttattgaagttttctttattatcttaacacgagatggaggtcagagtgtcagaccgggatctccatgatggagtataaaacattgatgttattaaactacgtagcatatgcctatttggactgaagattggatcaggatagtggttcagctgtacttccctcagaatcagtaagtataccgtatagcactcaacaaagcgaggcaacaggtttggagtattcgaacaagacataagtttggagtaatttgtaccggtggttgcaatcagagggggaaatggtgtgtgcaaccgttttctctataatagcatgttgggaaacacttttaatcgcctaggtggacatccacctatttgttgcatgtcaactaaataggtatgaaaaaaaatcaaaaaaattccagaaaaatagtagtaactgtatgctagtaagaggattggttcccataggtgtaaccaaacagcaaagataccaccagactgatcggtgcttgtgagcgagctcaagaagaactggacatctgaacattcaactgattgcttccagctatggaataacctgaagaaggaaaatggactgtgttatacatacattgctgttctctctctttttttttaacagggaattgaatttctctgtctctcagaaaattatctgttaaacctgtagttgttctcgaaaataaaaactgtagacctcaatgtaacaagatgttacccatggaagatggatgagagcatttctcccgatgaacaacaaaataatagagtggaaagttcctgatcacgacttcacgagttacagcaaactaaacaagagctgggtccaagatgtttcagtttcttccgatttctcggatccatgtctccactctccacacattgttaatgttctcaacttaacagaagaatcatttttacagcaagtgatcaatgaaaaataatagtatctgacaatgatattcttttgaaaaaagaaaatattcgaccctacataggcacatcatttgagatctggaagttgaaagaaaatgataacagacagtgatatcaattcgtatgcgtactagctaagacgagttggcgtgacaagattggaaacatattgaacacccgcagaaaagcactgtgtaaaaactgaaacgtgttatactctacagaaacaggacaaccgactctgaaagatgcaccgattctctgatttttttgcaaggctattctgtgacctgaaatttcagtcacctatgctgtacacggtgctttcattttccccctgtaatatccattttctgctcgaaacattggtcctattaaatcaacctctgaccatgactaacatcacttcctgaacaatgattttcgtagaccaatccccccgaacactgtcgatcggaacaattcgacattctgcagtttcctcctgctgcaacactatctgaaaatcggaacgattcaggcaccgtatcgttgtctgattcgtcgattcgttccatcattagtgtgttcacaacgcaaagacgatgactgtagtagtatctgcgtgctgattacatccaaatacagaatgatcatctccgagaatgcgaaaggggggcaattgacgtagccgcccccgccgttgtttgcttccccatctcgcactcttccgagacagccacggcctacccactcacatcgcataaacaaaaaacgagtacttacaaccatacgagtatacaccataataccatatactccacccctgtatcccgataatgaaaacgaaatgtatgatatagtaggcatataattaaaacatgcaacgaacaaatagaatactagtagtagaatacacacacgcacattgcctgtgttgttacatgtgtacaacagcttaacctccaaaccccccatggaagctttgcagccttgcatgttactcatttgacccacccctcagtagtagtactaccctctatctctctatatacaaaatgaaagggaatgagattcaattaagcagctagaacaggggggagtttacaagtgttggtggaggggaagagatcttgcaaccataaaagaagaaagcaggcgaaccaaaccaaacataccaaagggaagcaaagaaaagggcaaacatccagcaaaagtcaccaaacttgtaaagggatcgacacatatataggagagcgaggtgtccagtcgagaaggacagccaggccaaggagctcctagcctcctagcctacacagagctaagcaaagaggcaaagagctcagcaagaagctccattaatccattccatatactcatatagagaggaggaggaggaggaagaggaagagaggggaagcaaagcaagaagctttttttacttctcgggtttgcaagccaccgggaaaggccggggcttggtggtggtggtggtggtaagttttctttaaagcggcggagcgggaggccttcctgggcgctccggagatgcagcagcggcggaagtcggtgttcgcctcggcgccgttcgcgatgaagcaggcggcgctgggggccggcgtggcggcgcgcaggaacggcgcgccgctgtcgctggcggcggtggtgttcgcgctcttcgtgttcgcgacgttcctctacaacgaggacatcaagtccatcgccgacttccccttcggcgccggcgcgctccgcgccaagtcccccgacctccacgtcctccaggagaccgtgggcgccgcgcacctcgccgccggcagcatcgccaagcgcggcgaggaggtcatcgtccgtgtcctcgacgcgcccgcctccacggcgatggcggccgccgccggcagcagcagcaacaacagcacgattgaggtggccaaggccaatgccaacgccaacgccaacgccgccgacgccggtgtcaaggtggacgaggggcaggagagggagcgggacgtgacgctcccgagtgtcaaggaaggcggcgccgacgaggcgaggcggcgggaggacgaggaggccgccgagaaggagtcctcggcgaaggccgccgcggcgacggcggcgctgcggaccgtggtgagcgtgccggacacctgcgacctgtaccgcggcaactgggtgtacgacgaggtgaacgcgccggtgtacaaggagtcgcagtgcgagttcctgacggagcaggtcacctgcatgcgcaacggccgccgcgacgactcctaccagaagtggcgctggcagccgaccgactgcgacctgccccggtacgccatttcttcccccacaattttttcacgccatttcttgcccaatttcgtgccgtttcacgcaattccgggtaaccggcattgactcacgccggcgatcatcgtcggtttcccaaattgatgccggtttctacccttttctttttcttttttcatcagcatcggtcggtcacctcgaaacgctaagtcagaaataaccgtaggaatacttacgggccaaaattaagacgaacgtgtcagggaaattaaaatagaacgttcgagatttttgaaacggaacagagagtagtagtaccaccatgccacaccagtacaccactctggtagtgagctgtgacgtcggactgtactgtgcagccgcgctaccccctgttggattaagctttgcttaatctaatcactcagcatctggctgctgttgaagcagccactacttaaatcaaacacaggcacgccaaattattctcttttccaaccggggaacagctgcacgcagcgacgcccagtcagtcacacagtgggtgagtgacgcgagtagaatatactactacaacttgctctgtatctgtgtctcggcacagtctgatgatagtggcaaatatagttggcgatccaacaacgctggttgctaaataacgcaggaaaagtctaacgaaagctgatgtggtgtggggaaaaaaaaactgcaggttcgacgcgaggctgctgctggagcggctgcgcaacaagcggctgatgttcgtgggggattcgctgaaccgcaaccagtgggagtcgatggtgtgcctggtccagtccgtgatccccaagggcaagaagacgctcaccaagttcgtcaacggcggcaacagcaacatcttctacgcacacgtacgtagcaggcatcagcaacgtcacgtacgtactccgctgtcgatccgacatgatctccccctgacgatacggtgtatgtgctacgtgcaggagtacaacgcgacggtggagttctactgggcgccgttcctggtggagtcgaactcggacaacccgcaggtgcacagcgtccccgaccgcgtcatccagtggcactccatcgccaagcacgcgcacaactggctcggcgtcgactacctcatcttcaacacctacatctggtggctcaacaccctcgacatgaaagtcctgtaagttgtactcctagcaccccagcagcgtacacacatacatatagtgatatagaagtgggtgtgctgatctgggcgaaaaaatatgtgtgtggttgtacgtgacaggaaggggtcgttcgaccagggtgcgacggagtacgtggaggtggaccggcccgtggcgtacaaggaggtgctcaagacgtgggcgaagtgggtcgaccgcaacattgaccccaaccggacgaccgtcttcttcatgagcatgtctcccaaccacatcacgtaagccgccatttatgcctttgcctgcatctgcattgcattgcccctctccatgatttacttcgctgcacagtgcacacgtgcccacgcgcagtacagttcgctcggagttaatctaccaatcaataggaagatatgctgcacagtgatggtacatagagaactccattccgattaggtgttggggtatgttcagaaaacaaaattaggtgcttgaggtgtacctagcaactgtgttgcgtttgcaaggaggaaacttagctagcagccacacttgctttctggactaatagtaatctacaagtaatttaattaggtggtgagagcctgaggggactcgtgagctttcggagaaacattagtagtagaagcctcttatacattaactagctactagtagcatggtatcggaatacaactgcacatggtatcccatcatagagcactgtactactcatcggtagctgtctctcgtgaaatggcatcgtggggcacggtacctgcgtacaacgcgtcactgtacttcgtcaggacatggtccggacaatctgttctgactggttcaagatttgcatttttcacggtttcaacactggtatctaagcttctttagccaatactataaaaagcttaattaattagtgagatgtcttcaaaacatgctagttgacgtgggtgtagaaacaactgaggtttgttgaaccaagaataattcaaattgagtatgtatttgtgtagagtagtaagctcaaaactagtcatgtggatagctggagagaggcgagctacggtgacaagctgatgttggtggaccatagaaaatcccaggcccacctgtaggcatgcatctcgacatggcccaccctagtcgctaccaaagatgaagcatacgaatccaattccccgagctccatgtcactttccttcctcgagccctaaccccacgaaagagaaacgacatcaagttgaaggtagcaacaaaagacagtagtataatactgatagcaatcaagcctactaatcgtgctccacaatgatgatcagcctttgccattgcatctttgctgttgcaaaatactcaaaacagagagattcttgcagtagtcgacaagcaaaagttaaatcagttttggtccctgcaagaatctcgagctagtatccgcataaattacacggtccgtgcatatgctgtgataatggcgccaatatgcgagtgccatcatcatcagggtatcatcagcacgacagaatctagggtaccacggctccaattcgctggaacattaaccatagtactgcatcagaattctacccatcacggcatcacgtggtgacggacggatggtggtataatcttgtgtcgtgtgcgcgttttgtggcaggccggaggcgtggggaaactacggagggatcaagtgcgcgatggagacgctgccgatcacgaaccggacgacgtcgctggacgtgggcacggactggaggctgtacgccggggcgcaggaggtgctccagacgttccgccgggtgccggtgcacctcgtcgacatcacggcgctgtcggagctccgcaaggatgcgcacacgtcggtgcacacgctccggcaggggaagctgctcacccccgagcagcagtccgaccccaagacgtacgccgactgcatccactggtgcctcccgggcctccccgacacctggaaccagttcctctacgcccgcatcgcctccgccccatggtcatccgaccagtagaaaaagagccaaataaggctagcttccaagtgttctgcgagacaagaaaaggagccaagaacaccgttgacacaccatcgatgcccgccgcccggccgcggccacctcactttttcttactttgggttacttttttttcttctctttttttcaccatttttctttctttctctggggttgcgtaaagctgtcttgtgctgtgtttaacatgtgattcttttgttaaaaaagaaatggagggtttaggatggtgaaggaggatctgtgggcgggctgtgttgactatagctcatttcttgtcctgctgataaaggatgcatggataggagaaagcaaaaaaagggtgtgatgtgagatgtaaggggggaaagaaaaagggagggttacatgtaaagaatttttgtgtattacaccattctagtacattgatgctcaagagaggtataaaggccacgaggtgcatccgttcttcaattatgatgatgattcctaagtttctccttttcattttattacactgagtgatcacttcactccacctactgttccggaactactactatgcacgaatgttcagaaaagagattggcaatggaataacacagtctgcacttctctgaagataattgtaggggaataaagctccggaagcgtgcatagctcccctttctctttactcttatctttaggcaaactgtgagcaaagcaactcagctacattcattcagtctatacttttctgcacgcagggtcaaattacccagcaaaatctctggttaccaatagcatgagtgcatgacaaaggccatagccaaacgggactgcctatagatcatttgatagaaaatccctttagaaatcttacaaattttggaccacccgattgctttaagatttatgcatcaaccaaaccctataaaaccttctcctctcattccccctcacgccgccactcctcgttagcgtgagcgtattccgcgttgccgccgccacccggtcccgccggcgagccccccccctccctctcccccctccagccatcgaaggcaagatgctcccgtcccgtcgtgtctggcaactcctcgccgccggatccgccaccatcggccaccacaaggcacaagcttgctatgcccccgccaccaacactggcccaccgccctcctccatcccagcagctctggtgtcgccgcctcgccgcccacccgtcctgctacccaccgctaggccgctgcctcccacgaccatccatctaagcccggtgaactcccccttcttccccctccccctccccctcccatcccacctcaccccaccccaagagcctaattcattggccctccgccagagtttgggttgtcactgacaggggcataggagttggagaagaagcagctggagcacgaattgcgaagcacataaaatctgcaagatttagagatagatttttctgtc

SEQ ID NO: 2 - DNA, 1677 bp, Rice
atgcagcagcggcggaagtcggtgttcgcctcggcgccgttcgcgatgaagcaggcggcgctgggggccggcgtggcggcgcgcaggaacggcgcgccgctgtcgctggcggcggtggtgttcgcgctcttcgtgttcgcgacgttcctctacaacgaggacatcaagtccatcgccgacttccccttcggcgccggcgcgctccgcgccaagtcccccgacctccacgtcctccaggagaccgtgggcgccgcgcacctcgccgccggcagcatcgccaagcgcggcgaggaggtcatcgtccgtgtcctcgacgcgcccgcctccacggcgatggcggccgccgccggcagcagcagcaacaacagcacgattgaggtggccaaggccaatgccaacgccaacgccaacgccgccgacgccggtgtcaaggtggacgaggggcaggagagggagcgggacgtgacgctcccgagtgtcaaggaaggcggcgccgacgaggcgaggcggcgggaggacgaggaggccgccgagaaggagtcctcggcgaaggccgccgcggcgacggcggcgctgcggaccgtggtgagcgtgccggacacctgcgacctgtaccgcggcaactgggtgtacgacgaggtgaacgcgccggtgtacaaggagtcgcagtgcgagttcctgacggagcaggtcacctgcatgcgcaacggccgccgcgacgactcctaccagaagtggcgctggcagccgaccgactgcgacctgccccggttcgacgcgaggctgctgctggagcggctgcgcaacaagcggctgatgttcgtgggggattcgctgaaccgcaaccagtgggagtcgatggtgtgcctggtccagtccgtgatccccaagggcaagaagacgctcaccaagttcgtcaacggcggcaacagcaacatcttctacgcacacgagtacaacgcgacggtggagttctactgggcgccgttcctggtggagtcgaactcggacaacccgcaggtgcacagcgtccccgaccgcgtcatccagtggcactccatcgccaagcacgcgcacaactggctcggcgtcgactacctcatcttcaacacctacatctggtggctcaacaccctcgacatgaaagtcctgaaggggtcgttcgaccagggtgcgacggagtacgtggaggtggaccggcccgtggcgtacaaggaggtgctcaagacgtgggcgaagtgggtcgaccgcaacattgaccccaaccggacgaccgtcttcttcatgagcatgtctcccaaccacatcacgccggaggcgtggggaaactacggagggatcaagtgcgcgatggagacgctgccgatcacgaaccggacgacgtcgctggacgtgggcacggactggaggctgtacgccggggcgcaggaggtgctccagacgttccgccgggtgccggtgcacctcgtcgacatcacggcgctgtcggagctccgcaaggatgcgcacacgtcggtgcacacgctccggcaggggaagctgctcacccccgagcagcagtccgaccccaagacgtacgccgactgcatccactggtgcctcccgggcctccccgacacctggaaccagttcctctacgcccgcatcgcctccgccccatggtcatccgaccagtag

SEQ ID NO: 3 - protein, 558 aa, Rice
MQQRRKSVFASAPFAMKQAALGAGVAARRNGAPLSLAAVVFALFVFATFLYNEDIKSIADFPFGAGALRAKSPDLHVLQETVGAAHLAAGSIAKRGEEVIVRVLDAPASTAMAAAAGSSSNNSTIEVAKANANANANAADAGVKVDEGQERERDVTLPSVKEGGADEARRREDEEAAEKESSAKAAAATAALRTVVSVPDTCDLYRGNWVYDEVNAPVYKESQCEFLTEQVTCMRNGRRDDSYQKWRWQPTDCDLPRFDARLLLERLRNKRLMFVGDSLNRNQWESMVCLVQSVIPKGKKTLTKFVNGGNSNIFYAHEYNATVEFYWAPFLVESNSDNPQVHSVPDRVIQWHSIAKHAHNWLGVDYLIFNTYIWWLNTLDMKVLKGSFDQGATEYVEVDRPVAYKEVLKTWAKWVDRNIDPNRTTVFFMSMSPNHITPEAWGNYGGIKCAMETLPITNRTTSLDVGTDWRLYAGAQEVLQTFRRVPVHLVDITALSELRKDAHTSVHTLRQGKLLTPEQQSDPKTYADCIHWCLPGLPDTWNQFLYARIASAPWSSDQ

SEQ ID NO: 4 - DNA, 20 bp, Artificial Sequence
  The sequence is synthetized.
gaggaactggttccaggtgt

SEQ ID NO: 5 - DNA, 20 bp, Artificial Sequence
  The sequence is synthetized.
gtggcactagcacacacgat

SEQ ID NO: 6 - DNA, 20 bp, Artificial Sequence
  The sequence is synthetized.
ccaccctagtcgctaccaaa

SEQ ID NO: 7 - DNA, 20 bp, Artificial Sequence
  The sequence is synthetized.
gcacttgatccctccgtagt

SEQ ID NO: 8 - DNA, 20 bp, Artificial Sequence
  The sequence is synthetized.
aaagtgttggtgagcatagc

SEQ ID NO: 9 - DNA, 20 bp, Artificial Sequence
  The sequence is synthetized.
tttgtgtttggagagacgag

SEQ ID NO: 10 - DNA, 21 bp, Artificial Sequence
  The sequence is synthetized.
atgttcaaccttgtcccgact

SEQ ID NO: 11 - DNA, 20 bp, Artificial Sequence
  The sequence is synthetized.
taaagacggcagctatcact

SEQ ID NO: 12 - DNA, 18 bp, Artificial Sequence
  The sequence is synthetized.
cccgtgatttcctccgac

SEQ ID NO: 13 - DNA, 19 bp, Artificial Sequence
  The sequence is synthetized.
tcgctggttcgcttcatcg

SEQ ID NO: 14 - DNA, 21 bp, Artificial Sequence
  The sequence is synthetized.
tcaaatgttcaaagccgtaca

SEQ ID NO: 15 - DNA, 20 bp, Artificial Sequence
  The sequence is synthetized.
aaatggcatatgggctctgt

SEQ ID NO: 16 - DNA, 20 bp, Artificial Sequence
  The sequence is synthetized.
cctggtggttagcaaaaagc

SEQ ID NO: 17 - DNA, 20 bp, Artificial Sequence
  The sequence is synthetized.
gaaggcactgtcagctggat

SEQ ID NO: 18 - DNA, 20 bp, Artificial Sequence
  The sequence is synthetized.
aacgtgggaatttctagccc

SEQ ID NO: 19 - DNA, 20 bp, Artificial Sequence
  The sequence is synthetized.
gttttgggcctaaacgagtg

SEQ ID NO: 20 - DNA, 20 bp, Artificial Sequence
  The sequence is synthetized.
catactcaacacgcaatgcc

SEQ ID NO: 21 - DNA, 20 bp, Artificial Sequence
  The sequence is synthetized.
tatctgcgacgacgactctg

SEQ ID NO: 22 - DNA, 20 bp, Artificial Sequence
  The sequence is synthetized.
agtggcctacccgacaaagt

SEQ ID NO: 23 - DNA, 20 bp, Artificial Sequence
  The sequence is synthetized.
aaagcttttgggctcctctc

SEQ ID NO: 24 - DNA, 28 bp, Artificial Sequence
  The sequence is synthetized.
cggaattcttcacttttgggcattgttc

SEQ ID NO: 25 - DNA, 28 bp, Artificial Sequence
  The sequence is synthetized.
cgggtaccctccggagcgcccaggaagg

SEQ ID NO: 26 - DNA, 28 bp, Artificial Sequence
  The sequence is synthetized.
cgggtaccatgcagcagcggcggaagtc

SEQ ID NO: 27 - DNA, 28 bp, Artificial Sequence
  The sequence is synthetized.
cgggatccctactggtcggatgaccatg

SEQ ID NO: 28 - DNA, 23 bp, Artificial Sequence
  The sequence is synthetized.
cctctacaacgaggacatcaagt

SEQ ID NO: 29 - DNA, 24 bp, Artificial Sequence
  The sequence is synthetized.
cctctatcaacgaggacatcaagt

SEQ ID NO: 30 - DNA, 22 bp, Artificial Sequence
  The sequence is synthetized.
cctctaaacgaggacatcaagt

(19) United States
(12) Patent Application Publication
(10) Pub. No.: US 2024/0240265 A1
(43) Pub. Date: Jul. 18, 2024

(54) MUTATION SITE OF IDEAL BRITTLE CULM MUTANT ibc IN RICE, CONTROLLING GENE IBC, AND USE THEREOF

(71) Applicants: HEFEI INSTITUTES OF PHYSICAL SCIENCE, CHINESE ACADEMY OF SCIENCES, Hefei (CN); WIN-ALL HI-TECH SEED CO., LTD., Hefei (CN)

(72) Inventors: Yafeng YE, Hefei (CN); Binmei LIU, Hefei (CN); Conghe ZHANG, Hefei (CN); Yuejin WU, Hefei (CN); Liangzhi TAO, Hefei (CN); Zhi YAN, Hefei (CN); Libin HE, Hefei (CN); Guangle SHEN, Hefei (CN); Hui WANG, Hefei (CN); Yue ZHAN, Hefei (CN)

(73) Assignees: HEFEI INSTITUTES OF PHYSICAL SCIENCE, CHINESE ACADEMY OF SCIENCES, Hefei (CN); WIN-ALL HI-TECH SEED CO.,LTD., Hefei (CN)

(21) Appl. No.: 18/574,729

(22) PCT Filed: May 16, 2022
(86) PCT No.: PCT/CN2022/092957

§ 371 Date: Dec. 27, 2023

(30) Foreign Application Priority Data
Aug. 31, 2021 (CN) 202111013421.4

(51) Int. Cl. C12Q 1/6895; C12Q 1/686; G01N 27/447

(52) U.S. Cl. CPC C12Q 1/6895; C12Q 1/686; G01N 27/447; C12Q 2600/13

(57) ABSTRACT

A mutation site of an ideal brittle culm mutant ibc in rice, a controlling gene IBC, and use thereof are provided. The controlling gene IBC has: (1) a nucleotide sequence shown in SEQ ID NO: 1 or SEQ ID NO: 2; or (2) a nucleotide sequence of a mutant, an allele, or a derivative generated by adding, substituting, inserting or deleting one or more nucleotides. The present disclosure further provides an encoded protein of the controlling gene IBC, a recombinant construct, a recombinant host cell, a method for embrittling culms in a rice ripening stage by using the controlling gene IBC, and use thereof. In a mutant with allelic variation in the gene IBC and loss of gene editing function, culms exhibit a brittle culm phenotype at the late ripening stage, leaves are not brittle, yield traits are excellent, and other agronomic traits are not obviously changed.

### CROSS REFERENCE TO THE RELATED APPLICATIONS

This application is the national phase entry of International Application No. PCT/CN2022/092957, filed on May 16, 2022, which is based upon and claims priority to Chinese Patent Application No. 202111013421.4, filed on Aug. 31, 2021, the entire contents of which are incorporated herein by reference.

### SEQUENCE LISTING

The instant application contains a Sequence Listing which has been submitted in ASCII format via EFS-Web and is hereby incorporated by reference in its entirety. Said ASCII copy is named GBHZY019-PKG_Sequence_Listing.txt, created on 10/12/2023, and is 24,088 bytes in size.

### TECHNICAL FIELD

The present disclosure relates to the field of biotechnology, and in particular to a mutation site of an ideal brittle culm mutant ibc in rice, a controlling gene IBC, and use thereof.

### BACKGROUND

Rice (Oryza sativa L.) is one of the most important food crops in the world, and the mechanical strength of its culm is one of the important agronomic traits, which is directly related to the lodging resistance of rice plants, thus ultimately affecting the yield of rice. In another aspect, rice also produces a large amount of straw every year, which can be used for livestock feed, papermaking raw materials, building materials, biomass energy, organic fertilizer raw material and others. However, due to the anti-degradation barrier of straw biomass, it is very difficult for straw to return to the field and other comprehensive utilization. It is highly desirable to break through the bottleneck of comprehensive utilization of rice straw from the composition and structure of rice straw itself.

The mechanical strength of rice culms and their biomass anti-degradation barriers are determined by the secondary cell wall. The secondary cell wall is a cell wall layer that continues to accumulate on the inner side of the primary cell wall after specific cells stop growing. The secondary cell wall is essentially composed of cellulose, hemicellulose, and lignin. The change of each component will affect the structural changes of the secondary cell wall, thus affecting the mechanical strength of the plant. In rice, brittle culm mutants are important materials for studying the biosynthesis of the secondary cell wall in rice. Most of the reported brittle culm mutants eventually affect the thickening of the secondary cell wall due to the decrease in cellulose content. In recent years, researchers in China and other countries have cloned many key genes for controlling the synthesis of the secondary cell wall in rice. For example, OsCESA4, OsCESA7, and OsCESA9 encode the catalytic subunit of cellulose synthase in the secondary cell wall of rice. Their mutations will lead to changes in culm mechanical strength, exhibiting a brittle culm phenotype. Among them, OsCESA9 has mutated from aspartic acid to asparagine at amino acid 387, exhibiting a semi-dominant brittle culm phenotype. Specifically, for example, Chinese Patent CN110964733A discloses a rice semi-dominant brittle culm controlling gene SDBC1, but the rice culms and leaves expressed by the gene can be easily broken. In addition, there are the BC1 and BC12 genes that affect the assembly and arrangement of cellulose, the BC3 gene that affects the transport of cellulose synthase vesicles, the BC10 gene that affects the hemicellulose synthesis, and the CEF1 gene that regulates the synthesis of the secondary cell wall in rice. Research on these genes provides a new theory for the synthesis of secondary cell wall in rice, and these mutant materials provide new genetic resources for the efficient utilization of rice straw.

In order to realize the efficient comprehensive utilization of rice straw, in addition to changing the composition of rice culm, it is further necessary to take into account its yield-related traits. The efficient utilization of rice straw cannot be improved at the expense of rice yield. Although the changes in the cell wall composition of most rice brittle culm mutants have a very high potential for promoting efficient utilization of rice straw, the planting of rice brittle culm mutants faces lots of problems in the actual production process, for example, the leaves are easy to break, affecting the field operation; the culm is susceptible to lodging, affecting the final yield; and the like.

The ideal brittle culm rice suitable for large-scale planting and production needs to have the following characteristics: 1) excellent yield traits; 2) strong lodging resistance; 3) not brittle leaves; and 4) the culm becomes brittle at the late ripening stage.

### SUMMARY

One of the technical problems to be solved by the present disclosure is that leaves of a rice brittle culm mutant in the prior art are easy to break, affecting the field operation. The present disclosure provides a mutation site of a rice ideal brittle culm mutant ibc that has unbreakable leaves and is non-lodging, a controlling gene IBC, an encoded protein of the controlling gene IBC, a recombinant construct of the controlling gene IBC, a recombinant host cell of the controlling gene IBC, a method for embrittling culms at the rice ripening stage by using the controlling gene IBC, and use of brittle culm rice or brittle straw as a raw material in a feed or fertilizer.

The present disclosure solves the above technical problem by the following technical means:

A mutation site of an ideal brittle culm mutant ibc in rice is provided. The mutation site of the ideal brittle culm mutant ibc in rice is a chromosomal segment inversion, and the chromosomal segment inversion is located on an LOC_Os03g18140 gene.

The LOC_Os03g18140 gene is a controlling gene IBC.

A controlling gene IBC of an ideal brittle culm mutant ibc in rice is provided. The controlling gene IBC has: (1) a nucleotide sequence shown in SEQ ID NO: 1 or SEQ ID NO: 2; or (2) a nucleotide sequence of a mutant, an allele, or a derivative generated by adding, substituting, inserting, or deleting one or more nucleotides.

Alternatively, the nucleotide sequence shown in SEQ ID NO: 2 can hybridize under rigorous conditions, and simultaneously encode a nucleotide sequence controlling a culm mechanical strength at the ripening stage of rice.

The rigorous conditions are as follows: a hybrid membrane is placed in a pre-hybridization buffer (0.25 mol/L sodium phosphate buffer, pH 7.2, and 7% sodium dodecyl sulfate (SDS)) and pre-hybridized at 65° C. for 30 min; the pre-hybridization buffer is discarded, a hybridization buffer (0.25 mol/L sodium phosphate buffer, pH 7.2, 7% SDS, and isotope-labeled nucleotide fragment) is added, and hybridization is performed at 65° C. for 16 h; the hybridization buffer is discarded, a stripping buffer I (20 mmol/L sodium phosphate buffer, pH 7.2, 0.1% SDS) is added, and the hybrid membrane is washed twice at 65° C. for 10-15 min each time; a stripping buffer II (10 mmol/L sodium phosphate buffer, pH 7.2, 0.1% SDS) is added, and the hybrid membrane is washed at 65° C. for 10-15 min.

The nucleotide sequence shown in SEQ ID NO: 1 relates to a promoter, a coding region, and a flanking region of the controlling gene IBC.

In the present disclosure, heavy ions are utilized to irradiate a Chinese japonica rice cultivar, Oryza sativa subsp. japonica cv. Wuyunjing 7 (WYJ7), to obtain an ideal brittle culm (ibc) mutant having no effect on yield-related agronomic traits including plant height, tiller number, grain number per spike, thousand-grain weight, and lodging resistance, and exhibiting a brittle culm phenotype in the straw at the late filling stage.

In the present disclosure, through the genetic analysis and population construction of an ibc mutant and the analysis of genetic behavior thereof, it is found that the brittle culm phenotype of the ibc mutant is recessively controlled by a single gene IBC after mutation.

The present disclosure has the following beneficial effects: In the present disclosure, a gene IBC for controlling the rice culm mechanical strength is isolated and identified by map-based cloning; the phenotypic analysis and genetic complementation experiment of materials demonstrate the functions of the gene IBC in the regulation of mechanical strength and cell wall components of rice culms, and the gene is successfully used to cultivate a brittle culm rice cultivar Oryza sativa subsp. japonica cv. Kefujing 7 adapted to large-scale production, truly solving the problem of rice straw treatment from the source of the cultivar.

The IBC encodes xylan acetyltransferase and plays an important role in the modification of secondary cell wall hemicellulose polysaccharides. In a mutant with allelic variation in IBC and loss of gene editing function, culms exhibit a brittle culm phenotype in the late ripening stage, leaves are not brittle, yield traits are excellent, and other agronomic traits are not obviously changed; in the harvest stage, field-harvested culms are easy to crush, crushing and culm returning are facilitated, and the culms are easy to crush into silage in feed processing and to chew by animals.

The gene IBC provides a theoretical basis and material and gene support for elucidating the genetic basis for the regulation of rice secondary cell wall synthesis at the molecular level in the future, and the breeding of environmentally friendly new rice cultivars based on molecular design.

A protein encoded by a controlling gene IBC of an ideal brittle culm mutant ibc in rice is provided. The protein encoded by the controlling gene IBC has: (1) an amino acid sequence shown in SEQ ID NO: 3; (2) an amino acid sequence different from the amino acid sequence shown in SEQ ID NO: 3 due to the substitution, deletion and/or insertion of one or more (for example, 1-25, 1-20, 1-15, 1-10, 1-5, and 1-3) amino acid residues; or (3) one selected from amino acid sequences with at least 70%, preferably at least 80%, more preferably at least 90%, particularly at least 95%, 98%, or 99% identity with the amino acid sequence shown in SEQ ID NO: 3; or (4) one selected from active fragments of the amino acid sequences shown in (1), (2), or (3).

A recombinant construct is provided. The recombinant construct contains a nucleotide sequence of the controlling gene IBC for culm mechanical strength at the rice ripening stage, and a vector used for the recombinant construct is a cloning vector or an expression vector for expressing the nucleotide.

A recombinant host cell is provided, including a host cell of the foregoing recombinant construct, and a polynucleotide sequence integrated with the gene IBC for controlling rice culm mechanical strength according to the present disclosure in a genome thereof. The host cell may be selected from plant or microbial cells, for example, Escherichia coli or Agrobacterium cells, preferably the plant cells, and most preferably rice cells. The cells may be isolated, in vitro, cultured, or part of a plant.

The present disclosure provides a brittle culm rice cultivar obtained by using an ibc mutant and through a mutation site of ibc, and use of a brittle culm rice cultivar obtained by loss-of-function mutation in gene IBC by various means, including physical mutagenesis, chemical mutagenesis, biological mutagenesis and gene editing techniques, in straw treatment, including feed and fertilizer products using brittle straw as a raw material.

Preferably, the host cell is a microbial cell.

Preferably, the microbial cell is selected from an Escherichia coli cell and an Agrobacterium cell.

A method for embrittling cultivated rice culm is provided, including the following step: using a mutagenic means including physical mutagenesis, chemical mutagenesis and biological mutagenesis, or using a gene editing technique, to obtain a rice plant with loss-of-function of the foregoing controlling gene IBC of an ideal brittle culm mutant ibc in rice, where a resulting rice plant exhibits a brittle culm phenotype.

A method for embrittling cultivated rice culm is provided, including the following step: hybridizing an ideal brittle culm mutant ibc in rice having the foregoing mutation site with other rice cultivars, and segregating posterities to obtain a rice plant with a brittle culm phenotype.

The present disclosure provides a brittle culm rice cultivar obtained by using an ibc mutant and through a mutation site of ibc, and use of a brittle culm rice cultivar obtained by loss-of-function mutation in gene IBC by various means, including the foregoing physical mutagenesis, chemical mutagenesis, biological mutagenesis and gene editing techniques, in straw treatment, including feed and fertilizer products using brittle straw as a raw material.

Use of a brittle culm rice cultivar obtained by the foregoing method or brittle straw as a raw material in a feed or fertilizer is provided.

The present disclosure has the following beneficial effects: The brittle culm rice or brittle straw in the present disclosure n is used as a raw material, which is beneficial to chewing and digestion as a feed, and is easy to degrade in the field as a fertilizer.

A second technical problem to be solved by the present disclosure is to provide a method for identifying a mutation site of a controlling gene IBC for culm mechanical strength at the rice ripening stage.

The present disclosure solves the above technical problem by the following technical means:

A method for identifying the foregoing mutation site of an ideal brittle culm mutant ibc in rice is provided, including the following steps:
step 1, performing PCR amplification by using primers ibc-jd-1 and ibc-jd-2, where a forward primer ibc-jd-1-F of the ibc-jd-1 has a nucleotide sequence shown in SEQ ID NO: 4, and a reverse primer ibc-jd-1-R of the ibc-jd-1 has a nucleotide sequence shown in SEQ ID NO: 5; a forward primer ibc-jd-2-F of the ibc-jd-2 has a nucleotide sequence shown in SEQ ID NO: 6, and a reverse primer ibc-jd-2-R of the ibc-jd-2 has a nucleotide sequence shown in SEQ ID NO: 7; and
step 2, subjecting amplified products of the primers ibc-jd-1 and ibc-jd-2 to agarose gel electrophoresis detection, where detection results show that: if only the primer ibc-jd-1 has a target band, the mutation site of ibc is homozygous; if only the primer ibc-jd-2 has a target band, the mutation site of ibc is absent and a wild type is defined; and if both of the primers ibc-jd-1 and ibc-jd-2 have target bands, the mutation site of ibc is heterozygous.

The present disclosure has the following beneficial effects: Specific primers for PCR amplification are designed according to the unique mutation type of ibc, the primers are used for PCR amplification on rice DNA to be identified, and rice materials that are homozygous, heterozygous and free of the mutation site of ibc can be clearly distinguished through the agarose gel electrophoresis detection. The method can be used to track and identify the new brittle culm rice cultivars cultivated by using the mutation site of ibc.

The present disclosure has the following advantages: In the present disclosure, a gene IBC for controlling the rice culm mechanical strength is isolated and identified by map-based cloning; the phenotypic analysis and genetic complementation experiment of materials demonstrate the functions of the gene IBC of the ideal brittle culm phenotype in the regulation of mechanical strength and cell wall components of rice culms, and the gene is successfully used to cultivate a brittle culm rice cultivar Oryza sativa subsp. japonica cv. Kefujing 7 adapted to large-scale production, truly solving the problem of rice straw treatment from the source of the cultivar.

The gene IBC encodes xylan acetyltransferase and plays an important role in the modification of secondary cell wall hemicellulose polysaccharides. In a mutant with allelic variation in gene IBC and loss of gene editing function, culms exhibit a brittle culm phenotype at the late ripening stage, leaves are not brittle, yield traits are excellent, and other agronomic traits are not obviously changed; in the harvest stage, field-harvested culms are easy to crush, crushing and culm returning are facilitated, and the culms are easy to crush into silage in feed processing and to chew and digest by animals.

The gene IBC provides a theoretical basis and material and gene support for elucidating the genetic basis for the regulation of rice secondary cell wall synthesis at the molecular level in the future, and the breeding of environmentally friendly new rice cultivars based on molecular design.

Specific primers for PCR amplification are designed according to the unique mutation type of ibc, the primers are used for PCR amplification on rice DNA to be identified, and rice materials that are homozygous, heterozygous and free of the mutation site of ibc can be clearly distinguished through the agarose gel electrophoresis detection. The method can be used to track and identify the new brittle culm rice cultivars cultivated by using the mutation site of ibc.

### BRIEF DESCRIPTION OF THE DRAWINGS

FIG. 1 illustrates a comparison of plant types of a wild type WT versus an ibc mutant in Example 1 of the present disclosure;

FIG. 2 illustrates a comparison of broken culms of a wild type WT versus an ibc mutant in Example 1 of the present disclosure;

FIG. 3 illustrates a comparison of broken leaves of a wild type WT versus an ibc mutant in Example 1 of the present disclosure;

FIG. 4 illustrates a determination result of the culm breaking-resistant strength of a wild type WT versus an ibc mutant in Example 1 of the present disclosure;

FIG. 5 illustrates a determination result of the leaf breaking-resistant strength of a wild type WT versus an ibc mutant in Example 1 of the present disclosure;

FIG. 6 illustrates a plant height measurement result of a wild type WT versus an ibc mutant in Example 1 of the present disclosure;

FIG. 7 illustrates a tiller number measurement result of a wild type WT versus an ibc mutant in Example 1 of the present disclosure;

FIG. 8 illustrates a grain number per spike measurement result of a wild type WT versus an ibc mutant in Example 1 of the present disclosure;

FIG. 9 illustrates a panicle length measurement result of a wild type WT versus an ibc mutant in Example 1 of the present disclosure;

FIG. 10 illustrates a determination result of the seed setting rate of a wild type WT versus an ibc mutant in Example 1 of the present disclosure;

FIG. 11 illustrates a thousand-grain weight measurement result of a wild type WT and versus an ibc mutant in Example 1 of the present disclosure;

FIG. 12 shows scanning electron micrographs of cross sections of top second culms of a wild type WT versus an ibc mutant in Example 1 of the present disclosure;

FIG. 13 is a map of a gene IBC in Example 2 of the present disclosure;

FIG. 14 illustrates a MutMap analysis of a gene IBC in Example 2 of the present disclosure;

FIG. 15 is a structural schematic diagram of a mutation site of ibc in Example 2 of the present disclosure;

FIG. 16 illustrates an identification result of a mutation site of ibc in Example 2 of the present disclosure;

FIG. 17 is a structural diagram of a pIBCF expression vector in Example 2 of the present disclosure;

FIG. 18 illustrates a phenotype of a genetic complementary plant in Example 2 of the present disclosure;

FIG. 19 is a sequencing map of a gene IBC edited by CRISPR/Cas9 in Example 3 of the present disclosure; where the nucleotide sequence the WT is: CCTCTACAACGAGGACATCAAGT, as shown in SEQ ID NO: 28; the nucleotide sequence the ibc-c 1 is: CCTCTATCAACGAGGACATCAAGT, as shown in SEQ ID NO: 29; the nucleotide sequence the ibc-c 2 is: CCTCTAAACGAGGACATCAAGT, as shown in SEQ ID NO: 30;

FIG. 20 illustrates a brittle culm phenotype of a CRISPR Cas9-edited plant in Example 3 of the present disclosure;

FIG. 21 illustrates a plant type of CRISPR Cas9-edited plants in Example 3 of the present disclosure;

FIG. 22 illustrates chopped straw after wild type WT and ibc mutant materials are harvested in Example 4 of the present disclosure; and

FIG. 23 illustrates a comparison of straw chopping lengths of wild type WT and ibc mutant materials in Example 4 of the present disclosure.

### DETAILED DESCRIPTION OF THE EMBODIMENTS

In order to make the objectives, technical solutions and advantages of the examples of the present disclosure clearer, the technical solutions in the examples of the present disclosure will be clearly and completely described below with reference to the examples of the present disclosure. Obviously, the described examples are part of, not all of, the examples of the present disclosure. All other examples obtained by a person of ordinary skill in the art based on the examples of the present disclosure without creative efforts shall fall within the protection scope of the present disclosure.

All experimental materials and reagents used in the following examples may be commercially available, unless otherwise specified.

If specific techniques or conditions are not specified in the examples, the procedures shall be carried out in accordance with the techniques or conditions described in the literature in the art or in accordance with the product specification.

### Example 1

### Phenotypic Analysis of the Ideal Brittle Culm Mutant ibc

### (1) Analysis of Agronomic Traits

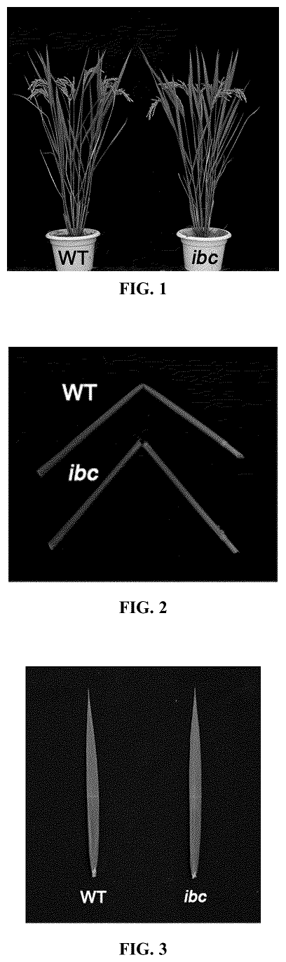

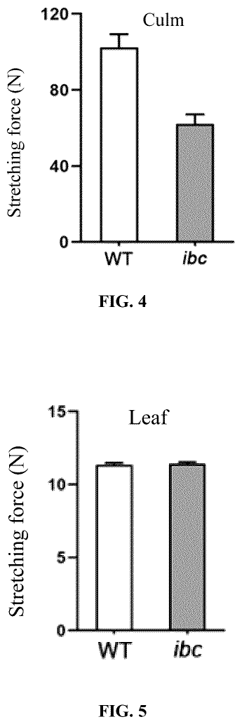

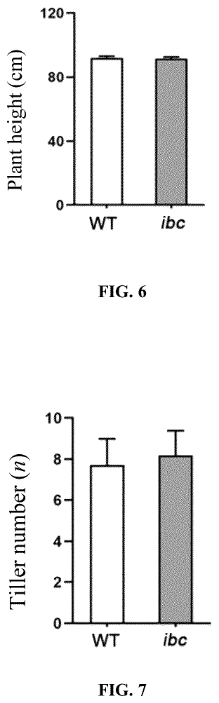

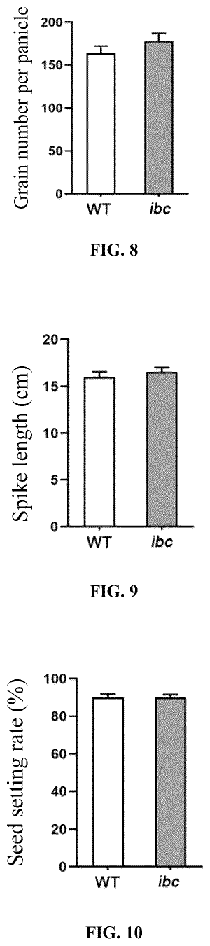

The ibc mutant was obtained by heavy ion ¹²C⁶⁺ mutagenesis (energy 80 MeV; dose 120 Gy) of japonica rice cultivar Oryza sativa subsp. japonica cv. Wuyunjing 7 (WYJ7). The phenotypic characteristics of the mutant were as follows: at the early heading stage, the plant height, tiller number and growth period were not significantly different from those of the wild type; at the late filling stage, the culm exhibited a brittle culm phenotype, and there were no significant differences in leaf and other agronomic traits, such as grain number per spike, panicle length, seed setting rate, and thousand-grain weight, as shown in FIGS. 1 to 11.

### (2) Genetic Analysis of the ibc Mutant

In order to investigate the molecular mechanism of ibc mutant forming a brittle culm at the late filling stage, the ibc mutant was first subjected to genetic analysis. Backcross populations were constructed by hybridization of the ibc mutant with wild-type Oryza sativa subsp. japonica cv. Wuyunjing 7. Among 480 plants in the F₂ segregating population, there were 112 plants with brittle culm phenotype and 368 plants with normal culms, and the segregation ratio of brittle culm plants to normal culm plants conformed to 1:3 (χ² [1:3]=0.18<χ² 0.05=3.84; P>0 05). The ibc mutant was hybridized with indica rice cultivar Oryza sativa subsp. indica cv. Huajingxian 74. Among 600 plants in the F₂ segregating population, there were 159 plants with brittle culm phenotype and 441 plants with normal culms, and the segregation ratio of brittle culm plants to normal culm plants conformed to 1:3 (χ² [1:3]=0.24<χ² 0.05=3.84; P>0.05). The above results indicated that the brittle culm trait of the ibc mutant was recessively controlled by a single gene pair and was not affected by the genetic background.

### (3) Scanning Electron Microscopy (SEM) of Cross Sections of Culms

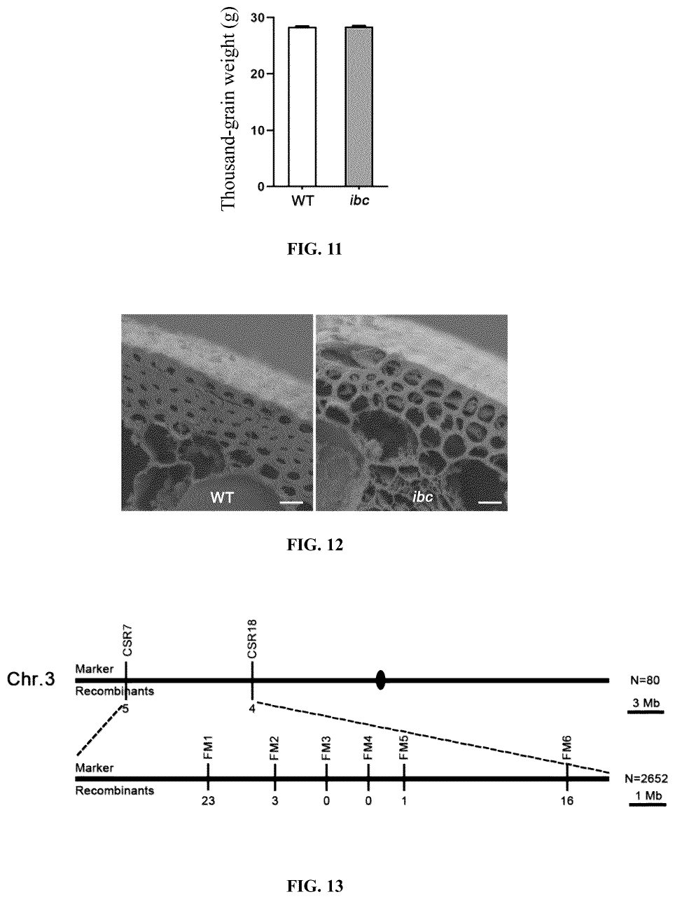

In order to further investigate the mechanism of the ibc mutant forming a brittle culm, the cross sections of the top second internodes of the wild type and the ibc mutant were observed by SEM. It was found that the cell wall of the sclerenchyma of the ibc mutant was significantly thinner than that of the wild type (FIG. 12), indicating that the thinning of the secondary cell wall of the sclerenchyma is the cause of the brittle phenotype of the ibc mutant.

### Example 2

### Gene Mapping of Ideal Brittle Culm Gene IBC

### (1) Construction of Mapping Population

The ibc mutant was hybridized with Oryza sativa subsp. indica cv. 93-11, Oryza sativa subsp. indica cv. Huajingxian 74, and Oryza sativa subsp. indica cv. Nanjing 11. The seeds of the segregating population were obtained by selfing the F₁ hybrids of different hybrid combinations obtained separately. These seeds were planted in the field. At the late filling stage, individual plants with brittle culm were selected as mapping individual plants. About 100 mg of leaves from each individual plant were used to extract DNA.

### (2) Screening of Simple Repeat Sequence (SSR) Polymorphism

The polymorphisms of the ibc mutant and Oryza sativa subsp. indica cv. Huajingxian 74 were screened to obtain polymorphic SSR primers for further experiment by using the reported SSR primers distributed uniformly on rice 12 chromosomes.

### (3) Gene IBC Mapping

Firstly, 21 random plants were selected from individual plants with brittle culm phenotype in the segregating population constructed by ibc mutant and Oryza sativa subsp. indica cv. Huajingxian 74 for preliminary mapping of gene IBC. The selected polymorphic SSR primers were used for linkage analysis of these 21 individual plants. The results showed that the molecular markers CSR7 and CSR18 on the long arm of chromosome 3 were significantly linked to the mutant gene. As shown in FIG. 13, the recombinants represent recons. The gene IBC was further determined to be located between CSR7 and CSR18, with an interval of about 8.66 Mb.

In order to further narrow the mapping interval of the gene IBC, more polymorphic primers were searched between CSR7 and CSR18 molecular markers (the primer sequences used for gene IBC mapping are shown in Table 1), and the number of mapping populations was expanded. Linkage analysis was performed on the larger mapping population. As shown in FIG. 13, the recombinants represent recons. Finally, the gene IBC was finely mapped between the Indel markers FM2 and FM5, approximately 3.40 Mb, and co-segregated from the molecular markers FM3 and FM4.

TABLE 1
Primers for gene IBC mapping

| Molecular | | | Physical position |
|---|---|---|---|
| marker | Sequence of forward primer | Sequence of reverse primer | (bp) |
| CSR7 | AAAGTGTTGGTGAGCATAGC, as | TTTGTGTTTGGAGAGACGAG, as | 3804124 |
| | shown in SEQ ID NO: 8 | shown in SEQ ID NO: 9 | |
| CSR18 | ATGTTCAACCTTGTCCCGACT, as | TAAAGACGGCAGCTATCACT, as | 12460908 |
| | shown in SEQ ID NO: 10 | shown in SEQ ID NO: 11 | |
| FM1 | CCCGTGATTTCCTCCGAC, as shown | TCGCTGGTTCGCTTCATCG, as | 5592412 |
| | in SEQ ID NO: 12 | shown in SEQ ID NO: 13 | |
| FM2 | TCAAATGTTCAAAGCCGTACA, as | AAATGGCATATGGGCTCTGT, as | 7105799 |
| | shown in SEQ ID NO: 14 | shown in SEQ ID NO: 15 | |
| FM3 | CCTGGTGGTTAGCAAAAAGC, as | GAAGGCACTGTCAGCTGGAT, as | 8678285 |
| | shown in SEQ ID NO: 16 | shown in SEQ ID NO: 17 | |
| FM4 | AACGTGGGAATTTCTAGCCC, as | GTTTTGGGCCTAAACGAGTG, as | 9889726 |
| | shown in SEQ ID NO: 18 | shown in SEQ ID NO: 19 | |
| FM5 | CATACTCAACACGCAATGCC, as | TATCTGCGACGACGACTCTG, as | 10503841 |
| | shown in SEQ ID NO: 20 | shown in SEQ ID NO: 21 | |
| FM6 | AGTGGCCTACCCGACAAAGT, as | AAAGCTTTTGGGCTCCTCTC, as | 11507516 |
| | shown in SEQ ID NO: 22 | shown in SEQ ID NO: 23 | |

### (4) Obtaining of Candidate Genes and Cloning of Gene IBC

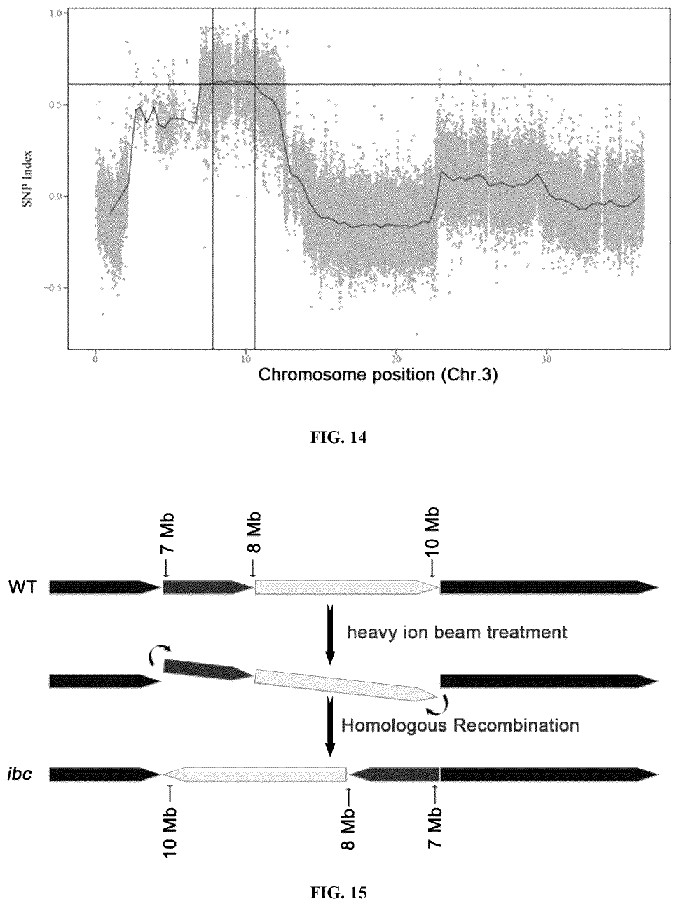

With the development of technologies such as next-generation sequencing (NGS) and third-generation sequencing (TGS) with longer read fragments, there is more selectivity in the mapping of mutant genes of different variation types. Using conventional map-based cloning techniques, the gene IBC cannot continue to narrow the mapping interval, which may be caused by its unique variation type. The gene IBC was considered to be mapped by NGS and TGS. The MutMap mapping results based on NGS were consistent with the above map-based cloning results (FIG. 14). Through the TGS and combined with the NGS results, the genome of the ibc mutant was assembled at a fixed point. The results showed that the ibc mutant had an inversion of a large chromosome segment in the above mapping interval (FIG. 15). The genes at both ends of the inversion were analyzed. It was found that one end of the breakpoint was located on the LOC_Os03g18140 gene, resulting in a loss-of-function mutation in this gene; the position of the other end of the breakpoint did not occur in the coding region of the gene. Therefore, LOC_Os03g18140 was used as a candidate gene for IBC.

### (5) Identification of the Mutation Site of ibc

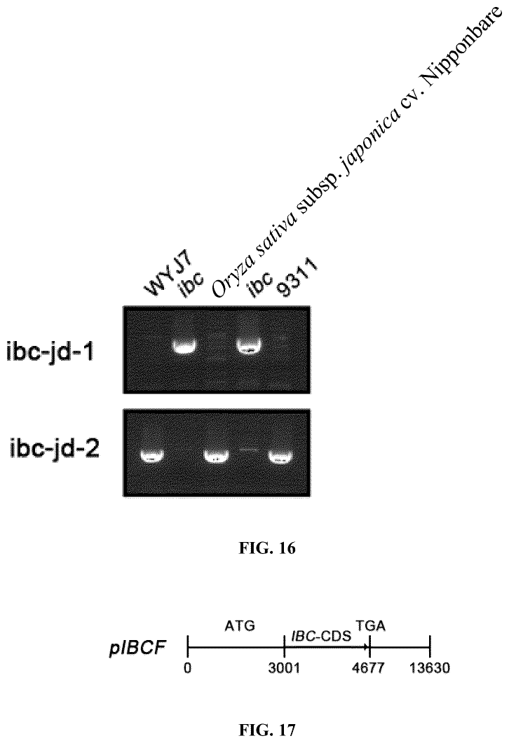

According to the type of ibc mutation, the corresponding identification primers were designed for PCR amplification. The primer names were ibc-jd-1 (the ibc mutation site could amplify the band, but the wild type could not) and ibc-jd-2 (the wild type could amplify the band, but the ibc mutation site could not), respectively. The identification results showed that the ibc mutant could only use the ibc-jd-1 primer to amplify the band, while the wild type could only use the ibc-jd-2 primer to amplify the band (FIG. 16).

### (6) Functional Complementary Verification of Gene IBC

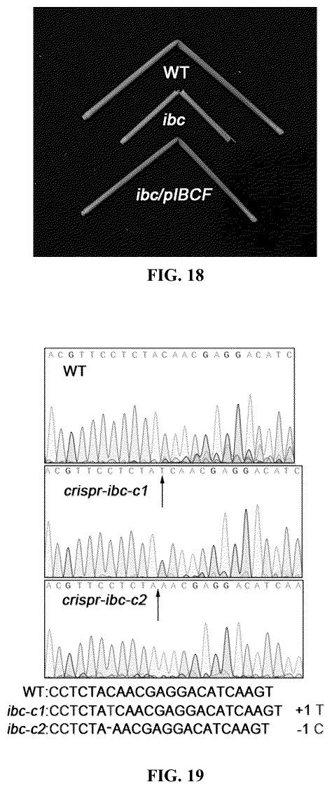

In order to verify that LOC_Os03g18140 is the gene IBC, RNA was extracted from the top second internode of the wild type as a material and cDNA was amplified by reverse transcription. An LOC_Os03g18140 expression vector pIBCF driven by LOC_Os03g18140 self-promoter (ATG upstream 3,000 bp fragment) was constructed. The structural diagram of the pIBCF expression vector is shown in FIG. 17. The pIBCF expression vector was transferred into the ibc mutant, and the culms of the positive transgenic T₀ plants obtained were all restored to normal levels, as shown in FIG. 18. This result proved that the LOC_Os03g18140 gene was the gene IBC.

The primers used to construct the expression vector pIBCF are as follows:

pIBC-F: 5′-cggaattcTTCACTTTTGGGCATTGTTC-3′, as
shown in SEQ ID NO: 24
pIBC-R: 5′-cgggtaccCTCCGGAGCGCCCAGGAAGG-3′, as
shown in SEQ ID NO: 25
IBC-CDS-F: 5′-cgggtaccATGCAGCAGCGGCGGAAGTC-3′, as
shown in SEQ ID NO: 26
IBC-CDS-R: 5′-cgggatccCTACTGGTCGGATGACCATG-3′, as
shown in SEQ ID NO: 27

The method for constructing the expression vector pIBCF included the following steps:

(1) The pIBC-F and pIBC-R primers were used to amplify the PCR product with the DNA of wild-type Oryza sativa subsp. japonica cv. Wuyunjing 7 as a template. The PCR product was double digested by restriction endonucleases EcoRI and KpnI, and the pCAMBIA2300 backbone was double digested by the enzymes. Then the amplified pIBC fragment was inserted into the pCAMBIA2300 vector by T4 ligase to obtain an intermediate vector pCAMBIA2300-pIBC.

(2) IBC-CDS-F and IBC-CDS-R primers were used to amplify the PCR product using the cDNA of wild-type Oryza sativa subsp. japonica cv. Wuyunjing 7 as a template. The PCR product was double digested by restriction endonucleases KpnI and BamHI, and the intermediate vector pCAMBIA2300-pIBC obtained in step (1) was double digested by the enzymes. The amplified IBC-CDS fragment was inserted into the pCAMBIA2300-pIBC vector by T4 ligase to obtain a final vector pCAMBIA2300-pIBC::IBC. The vector was pIBCF, as shown in FIG. 17.

### Example 3

### Method for Cultivating Ideal Brittle Culm Rice Cultivars

### (1) Cultivation of New Brittle Culm Cultivars by the Mutation Site of ibc

The ibc mutant was crossed, backcrossed and selfed with rice cultivars with normal culms, such as Oryza sativa subsp. indica cv. 93-11, Oryza sativa subsp. indica cv. Huajingxian 74, and Oryza sativa subsp. indica cv. Daohuaxiang 2, during which the mutation site of ibc and its genetic background were selected by using identification primers ibc-jd-1 and ibc-jd-2. Finally, a new brittle culm cultivar with homozygous ibc mutant gene under the background of Oryza sativa subsp. indica cv. 93-11, Oryza sativa subsp. indica cv. Huajingxian 74, and Oryza sativa subsp. indica cv. Daohuaxiang 2 was obtained. The specific implementation steps were as follows:

1. F₁ hybrids were obtained by crossing recipient parents, such as Oryza sativa subsp. indica cv. 93-11, Oryza sativa subsp. indica cv. Huajingxian 74, and Oryza sativa subsp. indica cv. Daohuaxiang 2, as male parents with the ibc mutant.

2. BC1F1 hybrids were obtained by backcrossing F₁ hybrid as a female parent with the recipient parents, such as Oryza sativa subsp. indica cv. 93-11, Oryza sativa subsp. indica cv. Huajingxian 74, and Oryza sativa subsp. indica cv. Daohuaxiang 2.

3. BC1F1 hybrids were planted, ibc genotypes were detected by using identification primers ibc-jd-1 and ibc-jd-2, respectively, the ibc heterozygous genotype was selected, namely, both PCR products of the above the primers ibc-jd-1 and ibc-jd-2 had target bands.

4. The genetic backgrounds of the individual plants selected in step 3 were identified by using molecular markers uniformly distributing on 12 pairs of rice chromosomes (including but not limited to SSR, SNP, InDel, EST, RFLP, AFLP, RAPD, and SCAR markers) and having polymorphisms between ibc mutant and recurrent parent, and the plants with high similarity to the genotype of the recurrent parent (such as more than 75%) were selected.

5. The plants selected in step 4 were backcrossed with the recipient parents, such as Oryza sativa subsp. indica cv. 93-11, Oryza sativa subsp. indica cv. Huajingxian 74, and Oryza sativa subsp. indica cv. Daohuaxiang 2, to obtain BC2F1 hybrids.

6. BC2F1 hybrids were planted, steps 3 and 4 were repeated, plants with heterozygous ibc genotype and high genetic background response rate (such as greater than 95%) were selected, and selfed BC2F2 seeds were harvested.

7. BC2F2 was planted, steps 3 and 4 were repeated, plants with heterozygous ibc genotype and the highest homozygous genetic background were selected, and BC2F3 hybrids were harvested. In ibc homozygous plants segregated from BC2F3 progenies, the detection results of identification primers ibc-jd-1 and ibc-jd-2 showed that only ibc-jd-1 could amplify a target band, and the culm phenotype was a brittle culm phenotype.

### (2) Cultivation of New Brittle Culm Cultivars Using Gene Editing Technique

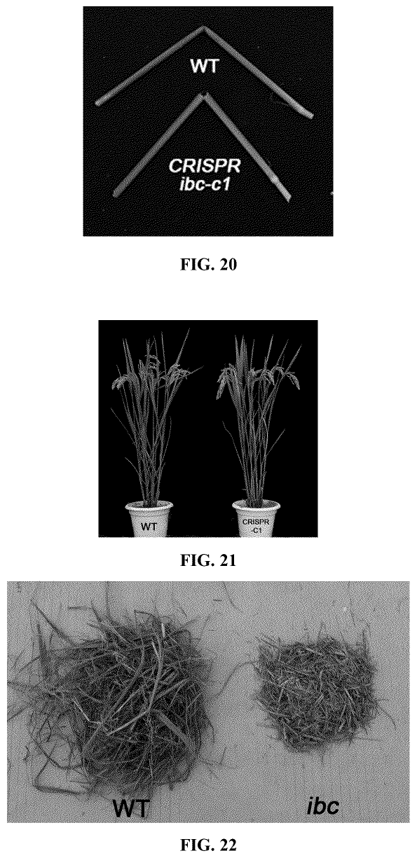

The gene IBC was edited by using the CRISPR/Cas 9 technology, and a mutant with loss-of-function of gene IBC was created. In several independent homozygous lines with gene IBC knockout, there were no significant differences in other agronomic traits (FIG. 21) except that the culms became brittle at the later stage (FIG. 20).

The construction and transformation method of the CRISPR/Cas 9 vector was as follows:

According to the gDNA sequence of the gene IBC, the target primer was designed as follows:

IBC-CRISPR-U3: 5′-CCTCTACAACGAGGACATCAAGT-3′, as
shown in SEQ ID NO: 28.

as shown in SEQ ID NO: 28.

The specific construction method was carried out with reference to the article (A robust CRISPR/Cas9 system for convenient, high-efficiency multiplex genome editing in monocot and dicot plants. (2015) Molecular Plant, 8(8):1274-1284) published by Prof. Liu Yaoguang in South China Agricultural University. The vector was introduced into WYJ7 by Agrobacterium-mediated transformation (rice transformation completed by the applicant's laboratory). Comparing the phenotypic analysis of wild-type and transgenic rice, it was found that in the crispr-ibc (the sequencing peak of the mutation site is shown in FIG. 19) of several IBC-knockout homozygous lines, the culm exhibited a brittle culm phenotype at the later stage (FIG. 20), and there was no significant difference in other agronomic traits (FIG. 21).

### Example 4

### Feed Processing Evaluation of ibc Brittle Culm Rice

### (1) Straw Field Chopping Experiment

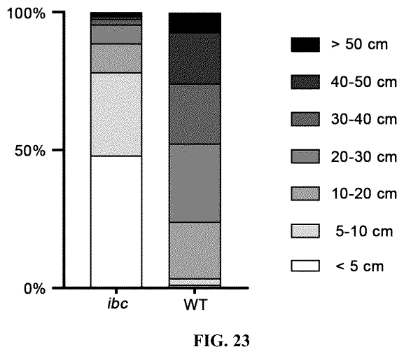

The wild type (WT) and ibc mutant materials were planted in a large area, and harvested in the field by a combine harvester during the harvest stage. As shown in FIG. 22, the straw of the ibc mutant was significantly better chopped after harvest than the wild type (WT). According to the statistics of the straw chopping length of a pair of materials, as shown in FIG. 23, the straw length of the ibc mutant after chopping is mostly concentrated in the length range of less than 5 cm and 5-10 cm. This length facilitates the later field degradation of the straw as a raw material for the organic fertilizer, and is also beneficial to the chewing and digestion of the cattle and sheep in the feed.

### (2) Goat Farming

In order to evaluate the feeding value of the brittle culm rice, research on the silage of brittle culm rice straw and the feeding of meat goats was carried out. A goat farm with more than 10 years of farming experience and a scale of more than 1,000 goats was selected for experimentation. Firstly, 5 tons each of brittle straw and ordinary straw were collected and processed according to the silage method of whole-plant corn, which was silaged in the form of large package, and the whole-plant corn silage in the same period was used as a control. Bags were opened for sampling and detection two months after silage.

The results showed that the brittle culm feed after silage appeared yellow-green, the sour flavor was very obvious, the texture was soft and non-sticky, and it had the characteristics of high-quality silage. Component detection showed that the content of soluble sugar and starch in silage brittle culm were higher than that of ordinary straw and whole-plant corn, showing excellent nutritional value. By selecting 36 goats of the same size, they were numbered by earmarking and divided into three feeding treatment groups. Each treatment was divided into three goatfolds, with 4 goats in each goatfold. According to the feeding method for using a certain proportion of roughage and fine grain, the goats were fed for two weeks during the pre-test and for two months during the formal test, and weighed every 20 days.

As shown in Table 2, the results show that the silage brittle culm feed has the best feeding effect, the overall weight gain rate is 48.6%, the average daily gain per goat is 137 g, which is 17.1% and 21.2% higher than that of silage corn and ordinary straw, respectively, showing a good application potential.

TABLE 2
The experimental results after feeding silage of brittle
culm rice, ordinary rice and whole-plant corn

| | | | | Net | | Weight |
|---|---|---|---|---|---|---|
| | | Initial | Feeding | weight | Daily | gain |
| | Quantity | weight | time | gain | gain | rate |
| Type | (n) | (kg) | (day) | (kg) | (g) | (%) |
| Brittle culm | 12 | 186.6 | 60 | 277.2 | 137 | 48.6 |
| rice | | | | | | |
| Ordinary | 12 | 199.0 | 60 | 280.6 | 113 | 41.0 |
| rice | | | | | | |
| Whole-plant | 12 | 193.1 | 60 | 278.0 | 117 | 43.9 |
| corn | | | | | | |

### (3) Cattle Breeding

Forty Holstein cattle: The test cattle were required to have normal growth and development with medium fat, be healthy and disease-free, and grow fast, aged around 4 months, weighing 140-185 kg. The cattle were randomized into four groups of 10 cattle: an ordinary rice straw group (group A), a brittle culm rice straw group (group B), a silage brittle culm rice straw group (group C), and a silage whole-plant corn stalk group (group D). They were fed in groups, twice a day (in the morning and evening). The concentrate limit was 40 kg/day/group, but ordinary rice straw and other test materials were not limited. The feeding time was 8 weeks.

Weight gain effect: The total weight gain was 737.5, 790.5, 825.5 and 841 kg for the ordinary rice straw group (group A), the brittle culm rice straw group (group B), the silage brittle culm rice straw group (group C), and the silage whole-plant corn stalk group (group D), respectively. It was indicated that the effect of the silage brittle culm rice straw was close to that of the silage whole-plant corn stalk, with the potential for alternative use.

Feed palatability: The highest feed intake was 1460.6 kg of silage brittle culm rice straw (group C), followed by 1429.4 kg of the silage whole-plant corn stalk (group D), and the worst was the ordinary rice straw (group A). The intake speed was as follows: silage brittle culm rice straw>silage whole-plant corn stalk>brittle culm rice straw>ordinary rice straw.

The foregoing examples are only intended to explain the technical solutions of the disclosure, and are not intended to limit the same. Although the present disclosure has been described in detail with reference to the foregoing embodiments, it should be understood by those of ordinary skill in the art that the present disclosure may still be modified with reference to the foregoing embodiments, or equivalent substitutions may be made to some of the technical features in the foregoing embodiments, and these modifications or substitutions do not make the nature of the corresponding technical solutions depart from the spirit and scope of the technical solutions of the embodiments of the present disclosure.

What is claimed is:

1-10. (canceled)

11. A method for identifying a mutation site of an ideal brittle culm mutant ibc in a rice, comprising the following steps:
step (1), performing a polymerase chain reaction (PCR) amplification by using primers ibc-jd-1 and ibc-jd-2, wherein a forward primer ibc-jd-1-F of the ibc-jd-1 has the nucleotide sequence shown in SEQ ID NO: 4, and a reverse primer ibc-jd-1-R of the ibc-jd-1 has the nucleotide sequence shown in SEQ ID NO: 5; a forward primer ibc-jd-2-F of the ibc-jd-2 has the nucleotide sequence shown in SEQ ID NO: 6, and a reverse primer ibc-jd-2-R of the ibc-jd-2 has the nucleotide sequence shown in SEQ ID NO: 7;
step (2), subjecting amplified products of the primers ibc-jd-1 and ibc-jd-2 to an agarose gel electrophoresis detection, wherein detection results show that: if only primer ibc-jd-1 has a target band, the mutation site of the ideal brittle culm mutant ibc is homozygous; if only primer ibc-jd-2 has a target band, the mutation site of the ideal brittle culm mutant ibc is absent and a wild type is defined; and if both of the primers ibc-jd-1 and ibc-jd-2 have target bands, the mutation site of the ideal brittle culm mutant ibc is heterozygous;
wherein the mutation site is a chromosomal segment inversion, and the chromosomal segment inversion is located on an LOC_Os03g18140 gene.
